(19) United States
(12) Patent Application Publication
(10) Pub. No.: US 2020/0323421 A1
(43) Pub. Date: Oct. 15, 2020

(54) ENDOSCOPE AND ENDOSCOPE SYSTEM

(71) Applicant: OLYMPUS CORPORATION, Tokyo (JP)

(72) Inventors: Suguru OKANIWA, Tokyo (JP); Seisuke TAKASE, Tokyo (JP); Hidehiro JOKO, Tokyo (JP); Isamu NAKAJIMA, Sagamihara-shi (JP)

(73) Assignee: OLYMPUS CORPORATION, Tokyo (JP)

(21) Appl. No.: 16/911,454

(22) Filed: Jun. 25, 2020

Related U.S. Application Data
(63) Continuation of application No. PCT/JP2018/029376, filed on Aug. 6, 2018.

(30) Foreign Application Priority Data
Dec. 28, 2017 (JP) 2017-253118

(51) Int. Cl. A61B 1/00; A61B 1/005

(52) U.S. Cl. CPC A61B 1/00135; A61B 1/00082; A61B 1/0052

(57) ABSTRACT

An endoscope according to the present invention includes an elongated insertion portion, a flexible tube included in a proximal end side of the insertion portion, a rigidity changing mechanism provided inside the flexible tube and configured to change rigidity of the flexible tube by operation on a hand side, and a large diameter portion provided on an outer circumference of the flexible tube and configured to indicate a distal end of the rigidity changing region of the flexible tube by the rigidity changing mechanism.

### CROSS REFERENCE TO RELATED APPLICATION

This application is a continuation application of PCT/JP2018/029376 filed on Aug. 6, 2018 and claims benefit of Japanese Application No. 2017-253118 filed in Japan on Dec. 28, 2017, the entire contents of which are incorporated herein by this reference.

### BACKGROUND OF THE INVENTION

### 1. Field of the Invention

The present invention relates to an endoscope including a rigidity changing mechanism in an insertion portion and an endoscope system provided with the endoscope and an overtube.

### 2. Description of the Related Art

Endoscopes provided with an image pickup unit to pick up an optical image within a distal end portion of an insertion portion, which can be inserted from an outside to an inside of a living body or a structure in order to observe places difficult to observe such as the inside of the living body or the structure are being used, for example, in medical and industrial fields.

An endoscope disclosed in Japanese Patent Application Laid-Open Publication No. 10-276965 includes a rigidity changing mechanism configured to change rigidity in a bending direction of a part of an insertion portion. The rigidity changing mechanism is provided with a coil pipe inserted in the insertion portion, a wire inserted in the coil pipe, and a traction mechanism configured to apply a compressive force to the coil pipe by pulling the wire. Rigidity in the bending direction of the coil pipe changes depending on the applied compressive force. For this reason, rigidity of the part of the insertion portion in which the coil pipe is inserted changes depending on the compressive force applied to the coil pipe.

Japanese Patent Application Laid-Open Publication. No. 2005-334474 discloses an endoscope system provided with an overtube to be externally fitted onto an insertion portion to assist an operation of inserting the endoscope into a subject.

In the endoscope disclosed in Japanese Patent Application Laid-Open Publication No. 10-276965, rigidity of only the region of the insertion portion in which the coil pipe is inserted changes. Thus, when rigidity of the insertion portion is increased by the rigidity changing mechanism, the rigidity of the insertion portion drastically changes between a rigidity changing region, which is a region where rigidity of the insertion portion changes and a boundary between the rigidity changing region and the region excluding the rigidity changing region (which is generally positioned between the distal end of the insertion portion and the bending portion).

If there is a spot where rigidity of the insertion portion drastically changes, the insertion portion is more likely to bend at the spot where rigidity of the insertion portion drastically changes when the insertion portion is inserted into a subject, which may interfere with the insertion operation.

Thus, for example, International Publication No. 2017/086312 discloses an endoscope system that combines an endoscope provided with a rigidity changing mechanism with an overtube. The endoscope system causes the whole overtube to move forward or backward along the insertion portion of the endoscope to make it possible to select whether to cover the boundary located at a front end of the rigidity changing region with the overtube or expose the boundary. This makes it possible to select between a state where the rigidity of the insertion portion is gently changed and a state where the rigidity is drastically changed at the boundary.

### SUMMARY OF THE INVENTION

An endoscope according to an aspect of the present invention includes an elongated insertion portion; a flexible tube included in a proximal end side of the insertion portion; a rigidity changing mechanism provided inside the flexible tube and configured to change rigidity of the flexible tube by an operation on a hand side; and an index provided on an outer circumference of the flexible tube, configured to indicate a distal end of a rigidity changing region of the flexible tube by the rigidity changing mechanism and made up of a large diameter portion having an outer diameter larger than an outer diameter of the flexible tube.

An endoscope system according to another aspect of the present invention includes an endoscope including an elongated insertion portion, a flexible tube included in a proximal end side of the insertion portion, and a rigidity changing mechanism provided inside the flexible tube and configured to change rigidity of the flexible tube, a rigidity changing region being set by the rigidity changing mechanism so as to extend from a middle portion of the flexible tube to a proximal end portion; a flexible, axially extending and cylindrical overtube, into which the insertion portion is slidably inserted, an overall length in an axial direction of the overtube being formed to be shorter than an overall length of the rigidity changing region, the overtube being configured to expose a distal end portion of the rigidity changing region when a proximal end portion of the overtube in the axial direction is positioned on a most proximal end side of the insertion portion of the endoscope; and a notification unit provided at a position corresponding to the distal end portion of the rigidity changing region in the flexible tube, made up of a large diameter portion having an outer diameter larger than an outer diameter of the flexible tube and configured to notify that the distal end of the overtube passes through the distal end of the rigidity changing region.

### BRIEF DESCRIPTION OF THE DRAWINGS

FIG. 1 is a diagram illustrating a configuration of an endoscope system;

FIG. 2 is a diagram illustrating an overtube externally fitted onto an insertion portion of the endoscope;

FIG. 3 is a diagram illustrating configurations of a flexible tube and a rigidity changing mechanism;

FIG. 4 is a diagram illustrating a positional relationship between the rigidity changing mechanism and the overtube;

FIG. 5 is a diagram illustrating a positional relationship between the rigidity changing mechanism and the overtube;

FIG. 6 is a diagram schematically illustrating a change in rigidity of the insertion portion in the endoscope system;

FIG. 7 is a diagram schematically illustrating a change in rigidity of the insertion portion in the endoscope system;

FIG. 8 is a diagram schematically illustrating a change in rigidity of the insertion portion in the endoscope system;

FIG. 9 is a diagram schematically illustrating a change in rigidity of the insertion portion in the endoscope system;

FIG. 10 is a diagram illustrating a first step of an insertion method using the endoscope system;

FIG. 11 is a diagram illustrating a second step of the insertion method using the endoscope system;

FIG. 12 is a diagram illustrating a third step of the insertion method using the endoscope system;

FIG. 13 is a diagram illustrating a fourth step of the insertion method using the endoscope system;

FIG. 14 is a diagram illustrating a fifth step of the insertion method using the endoscope system;

FIG. 15 is a diagram illustrating a sixth step of the insertion method using the endoscope system;

FIG. 16 is a diagram illustrating a seventh step of the insertion method using the endoscope system; and

FIG. 17 is a diagram illustrating an eighth step of the insertion method using the endoscope system.

### DETAILED DESCRIPTION OF THE PREFERRED EMBODIMENTS

Hereinafter, preferred embodiments of the present invention will be described with reference to the accompanying drawings. Note that in the drawings used in the following description, different scales are used for the respective components to illustrate the respective components in sizes recognizable on the drawings. However, the present invention is not limited only to quantities of the components, shapes of the components, size ratios among the components and relative positional relationships among the respective components described in the drawings.

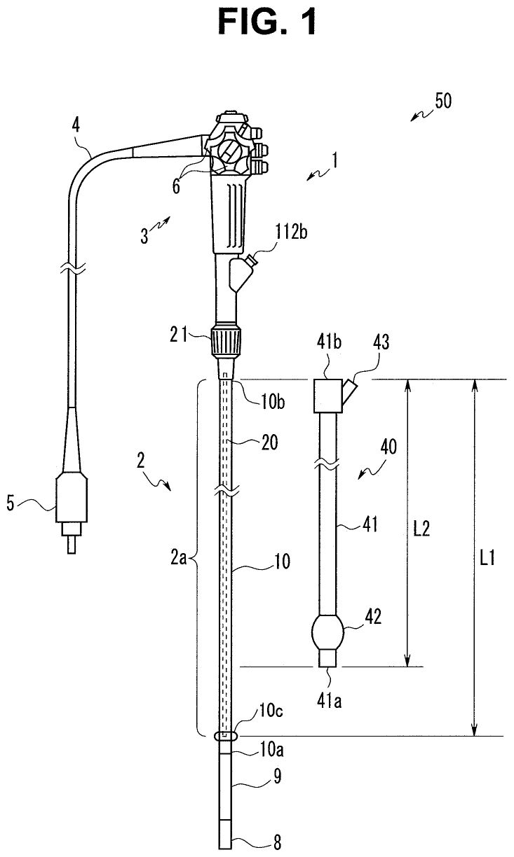

An endoscope system 50 of the present embodiment shown in FIG. 1 is provided with an endoscope 1 and an overtube 40. The endoscope 1 includes an elongated insertion portion 2 that can be introduced into a subject such as a human body, and the insertion portion 2 includes a configuration for observing the inside of the subject. Note that the subject into which the insertion portion 2 of the endoscope 1 is introduced is not limited to a human body, but may be other living bodies.

The endoscope 1 of the present embodiment is mainly constructed of the insertion portion 2 that is introduced into the subject and formed into an elongated shape, an operation portion 3 located at a proximal end of the insertion portion 2 and a universal cord 4 that extends from the operation portion 3.

The insertion portion 2 is constructed of a distal end portion 8 disposed at a distal end, a bendable bending portion 9 disposed on a proximal end side of the distal end portion 8, and a flexible tube 10 connecting a proximal end side of the bending portion 9 and a distal end side of the operation portion 3 and having flexibility, all of which are connected in series.

A configuration for observing the inside of the subject is disposed at the distal end portion 8. For example, an image pickup unit including an objective lens and an image pickup device for optically observing the inside of the subject is disposed at the distal end portion 8. An illumination light emitting section (not shown) of the image pickup unit that emits light for illuminating an object is also provided in the distal end portion 8. Note that an ultrasound transducer for acoustically observing the inside of the subject using ultrasound may also be disposed in the distal end portion 8.

The operation portion 3 disposed at the proximal end of the insertion portion 2 is provided with an angle operation knob 6 for operating the bending of the bending portion 9. An endoscope connector 5 configured to be connectable to an external apparatus (not shown) is provided at a proximal end portion of the universal cord 4. The external apparatus to which the endoscope connector 5 is to be connected is provided with a camera control unit or the like configured to control the image pickup unit provided in the distal end portion 8.

The operation portion 3 is provided with a rigidity changing knob 21 for operating the rigidity changing mechanism 20 disposed within the flexible tube 10. The rigidity changing mechanism 20 is inserted in the flexible tube 10 in the longitudinal direction of the flexible tube 10 and is configured such that rigidity against bending changes in accordance with operation input by the rigidity changing knob 21. In other words, the rigidity changing mechanism 20 causes rigidity against bending of the flexible tube 10 to change.

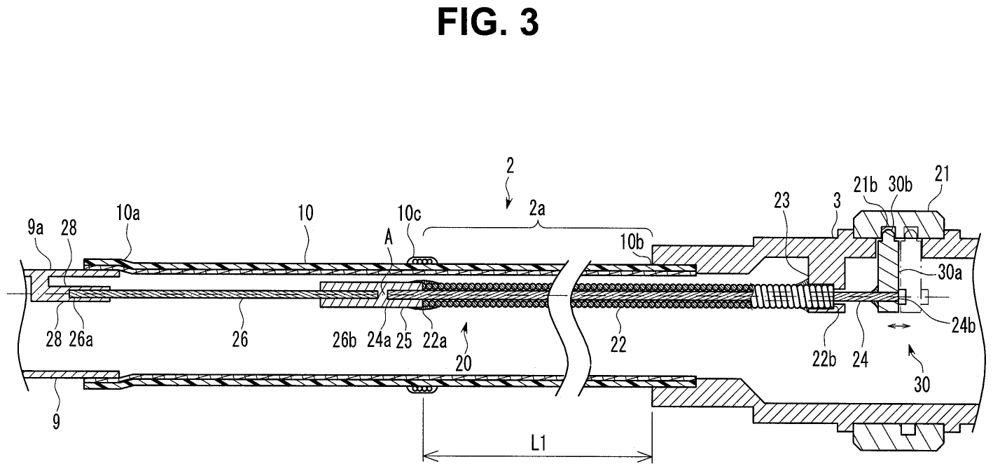

Since the configuration of the rigidity changing mechanism 20 is publicly known, although detailed description will be omitted, the rigidity changing mechanism 20 is provided with a coil pipe 22, a first wire 24, a second wire 26, and a traction mechanism 30 as shown in FIG. 3. Regarding members included in the insertion portion 2 and the rigidity changing mechanism 20, a direction toward the distal end portion 8 side of the insertion portion 2 is called a “distal end direction” and a direction toward the operation portion 3 side is called a “proximal end direction”.

The coil pipe 22 is a linear member formed by spirally winding a metallic linear elemental wire, for example, of a stainless alloy around a predetermined axis A parallel to the longitudinal direction of the insertion portion 2. A proximal end 22b of the coil pipe 22 is fixed to a coil fixing portion 23 provided inside the operation portion 3.

A distal end 22a of the coil pipe 22 is disposed within the flexible tube 10 at a position on the proximal end side by a predetermined distance with respect to a distal end 10a of the flexible tube 10. In other words, the coil pipe 22 extends within the flexible tube 10 from the proximal end 10b of the flexible tube 10 to a position before the distal end 10a of the flexible tube 10.

The first wire 24 is inserted in the coil pipe 22. A distal end 24a of the first wire 24 is fixed to the distal end 22a of the coil pipe 22 and a proximal end 24b is fixed to a wire holding portion 30a of the traction mechanism 30, which will be described later.

In the present embodiment, as an example, the distal end 24a of the first wire 24 is fixed to a connection portion 25, which is fixed to the distal end 22a of the coil pipe 22. Note that the distal end 24a of the first wire 24 may be directly fixed to the distal end 22a of the coil pipe 22.

A distal end 26a of the second wire 26 is fixed to a wire fixing portion 28 provided in a frame member 9a on a proximal end side of the bending portion 9, and a proximal end 26b is fixed to the connection portion 25. The second wire 26 restricts the movement of the distal end 22a of the coil pipe 22 within the flexible tube 10 in the proximal end direction and holds the position of the coil pipe 22 within the flexible tube 10 in the longitudinal direction. A large diameter portion 10c formed by winding a thread around an outer surface of the flexible tube 10 at a portion where the distal end portion of the coil pipe 22 is located inside and covering the thread-wound portion with an adhesive and configured to function as an index is disposed. An outer diameter of the large diameter portion 10c is slightly larger (e.g., larger by about 0.5 to 1.0 mm) than an inner diameter of a distal end portion 41a of a cylindrical portion 41 of the overtube 40.

The traction mechanism 30 is provided with the rigidity changing knob 21 configured to rotate with respect to the operation portion 3 and the wire holding portion 30a that holds the proximal end 24b of the first wire 24 and moves forward or backward along the axis A as the rigidity changing knob 21 rotates.

A cam groove 21b is carved on an inner surface of the rigidity changing knob 21. The wire holding portion 30a is provided with a cam pin 30b that slidably engages with the cam groove 21b. The engagement of the cam groove 21b with the cam pin 30b causes the wire holding portion 30a to move forward or backward along the axis A as the rigidity changing knob 21 rotates. The traction mechanism 30 of the present embodiment configured as described above pulls the first wire 24 in the proximal end direction as the user causes the rigidity changing knob 21 to rotate, and can thereby change tension applied to the first wire 24.

A compressive force is applied to the coil pipe 22 in accordance with tension applied to the first wire 24 by the traction mechanism 30. When the compressive force is applied to the coil pipe 22, a resistance force against bending deformation increases. Thus, rigidity of the flexible tube 10 against bending within a range in which the coil pipe 22 is disposed changes depending on the resistance force against bending deformation of the coil pipe 22. With the above-described configuration, the rigidity changing mechanism 20 changes rigidity of the portion of the flexible tube 10 in which the coil pipe 22 is inserted.

In the present embodiment, when the flexible tube 10 is linearly held, a length from the proximal end 10b of the flexible tube 10 to the distal end of the coil pipe 22 is L1. Therefore, a range of the length L from the proximal end 10b of the flexible tube 10 toward the distal end in the longitudinal direction is the rigidity changing region 2a where rigidity can be changed by the rigidity changing mechanism 20 in the insertion portion 2 of the endoscope 1 of the present embodiment.

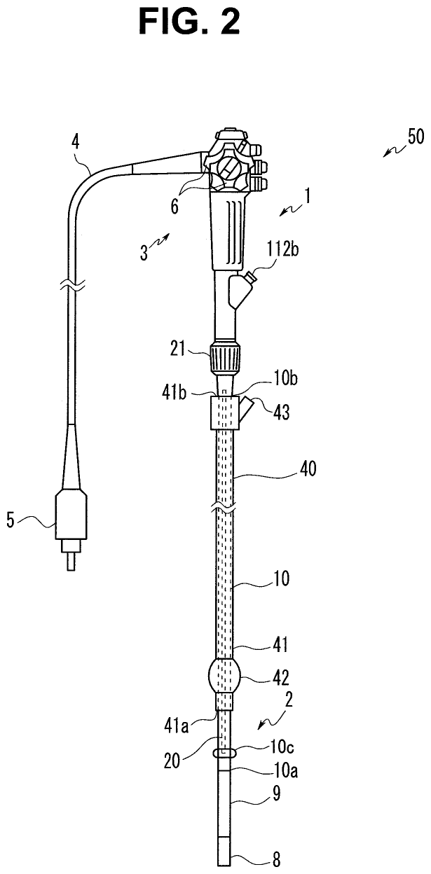

The overtube 40 is provided with a flexible, tubular cylindrical portion 41. The cylindrical portion 41 has a cylindrical shape, both ends of which are open, in which the insertion portion 2 of the endoscope 1 can be inserted as shown in FIG. 2. In other words, the cylindrical portion 41 can be placed on an outer circumference of the insertion portion 2. With the insertion portion 2 inserted, the cylindrical portion 41 bends according to the deformation of the insertion portion 2. The cylindrical portion 41 is relatively slidable with respect to the insertion portion 2 in the longitudinal direction of the insertion portion 2. An inner diameter of the cylindrical portion 41 in the distal end portion is such a diameter that allows the cylindrical portion 41 to slide with respect to an outer diameter of the aforementioned flexible tube 10, and is slightly smaller than the outer diameter at the large diameter portion 10c of the flexible tube 10 (e.g., smaller by about 0.5 mm to 1.0 mm). The inner diameter of the cylindrical portion 41 at the distal end portion is preferably set such that sliding resistance becomes as small as possible when the cylindrical portion 41 slides on the outer circumference of the flexible tube 10 and such that the operator's hand feels some resistance when the cylindrical portion 41 gets over the large diameter portion 10c.

FIG. 2 illustrates the cylindrical portion 41 disposed at a most proximal end with respect to the insertion portion 2. In other words, FIG. 2 illustrates the insertion portion 2 of the endoscope 1 pushed in the overtube 40 most.

As shown in FIG. 1 and FIG. 2, the length L2 of the cylindrical portion 41 in the axial direction (longitudinal direction) is shorter than the length L1 of the rigidity changing region 2a.

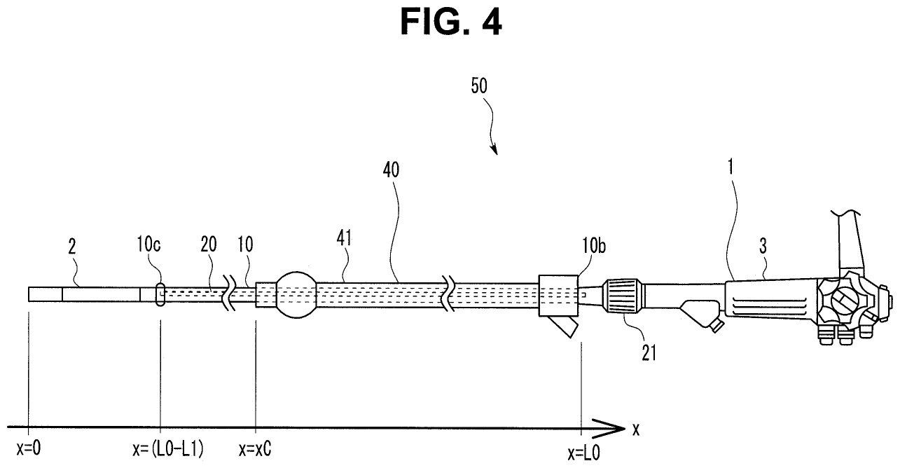

Therefore, as shown in FIG. 2 and FIG. 4, when the cylindrical portion 41 is disposed at the most proximal end with respect to the insertion portion 2, the distal end portion of the rigidity changing region 2a provided in the flexible tube 10 projects from the distal end portion 41a of the cylindrical portion 41 of the overtube 40 in the distal end direction. In other words, when the insertion portion 2 is pushed in the cylindrical portion 41 of the overtube 40 most, the distal end portion of the rigidity changing region 2a is exposed from the cylindrical portion 41 in the distal end direction.

A balloon 42 made of an extensible member is disposed at the distal end portion 41a of the cylindrical portion 41. Furthermore, a balloon vent 43 communicating with the inside of the balloon 42 via a pipe line (not shown) is disposed at a proximal end portion 41b of the cylindrical portion 41. The balloon 42 has a doughnut shape disposed so as to surround the outer circumference of the distal end portion 41a of the cylindrical portion 41. The balloon 42 inflates or deflates depending on inflow and outflow of gas through the balloon vent 43.

As described above, in the endoscope system 50 of the present embodiment, the overall length L2 of the cylindrical portion 41, which is externally fitted onto the insertion portion 2 of the overtube 40, is shorter than the length L1 of the rigidity changing region 2a of the insertion portion 2. Therefore, by changing the relative positions of the overtube 40 and the insertion portion 2 in the longitudinal direction with the overtube 40 externally fitted onto the insertion portion 2, the endoscope system 50 of the present embodiment can select a state in which the distal end portion of the rigidity changing region 2a is exposed from the overtube 40 in the distal end direction or a state in which the distal end portion of the rigidity changing region 2a is covered with the overtube 40.

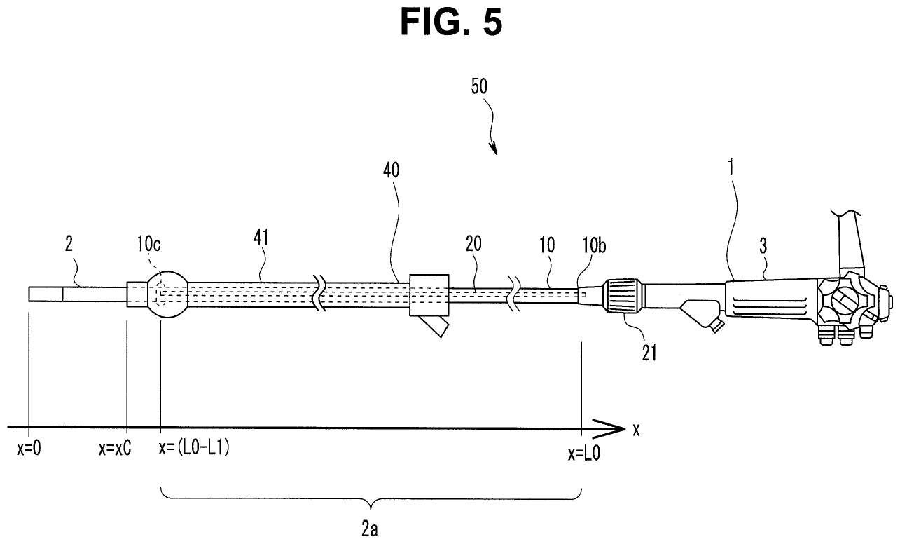

For example, as shown in FIG. 4, when the insertion portion 2 is relatively bushed in in the distal end direction with respect to the overtube 40, the distal end portion of the rigidity changing region 2a is exposed from the overtube 40 in the distal end direction. On the other hand, for example, as shown in FIG. 5, when the insertion portion 2 is relatively pulled back in the proximal end direction with respect to the overtube 40, the distal end portion of the rigidity charming region 2a is covered with the overtube 40. As shown in FIG. 4 to FIG. 5, when the insertion portion 2 is relatively pulled back in the proximal end direction, the distal end portion 41a of the cylindrical portion 41 of the overtube 40 gets over the large diameter portion 10c of the flexible tube 10 and advances to the distal end side of the insertion portion 2. In this way, the operator recognizes, from a feeling at hand, that the distal end portion 41a of the cylindrical portion 41 of the overtube 40 has moved toward the distal end side relative to the rigidity changing region 2a.

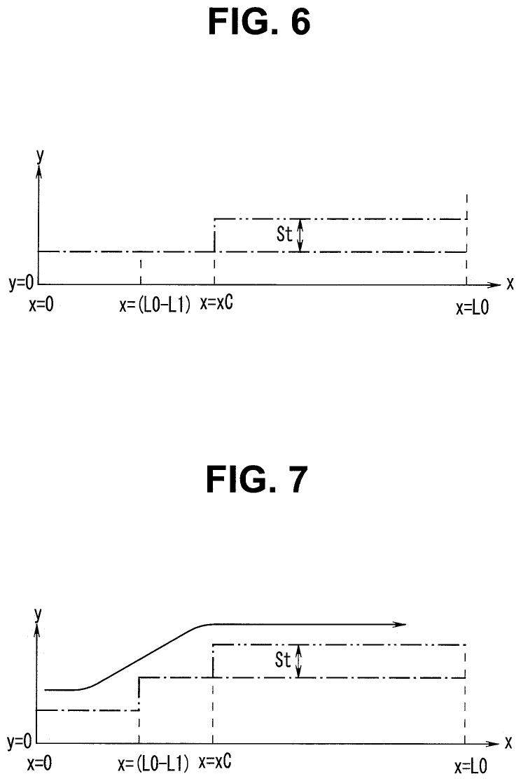

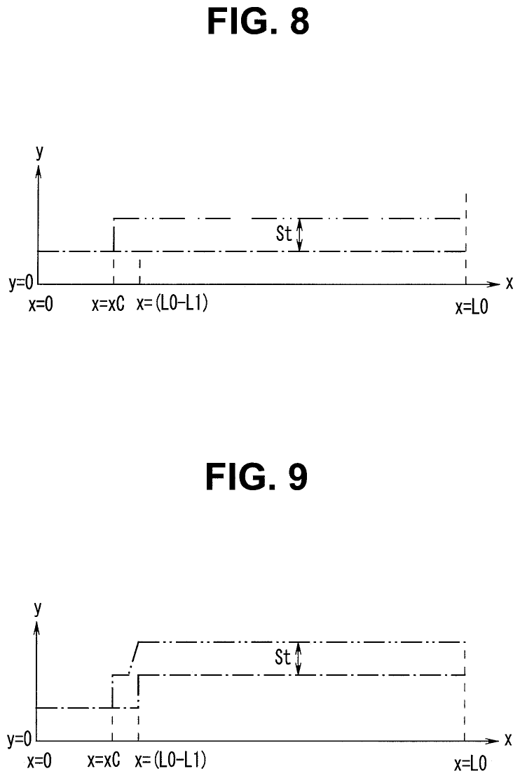

FIG. 6, FIG. 7, FIG. 8 and FIG. 9 illustrate a change in rigidity of the insertion portion 2 and the overtube 40 in the longitudinal direction. In schematic graphs shown in FIG. 6, FIG. 7, FIG. 8 and FIG. 9, an x axis, which is a horizontal axis, represents a distance from the distal end of the insertion portion 2 in the longitudinal direction. A y axis, which is a vertical axis, represents rigidity with respect to deformation of the insertion portion 2 and the overtube 40 in the bending direction.

On the x axis, x=0 represents the distal end of the insertion portion 2 and x=L0 represents a proximal end (proximal end 10b of the flexible tube 10) of the insertion portion 2. On the y axis, rigidity increases toward the top of the drawing. A single-dot dashed line in the drawing represents rigidity of the insertion portion 2 and a two-dot dashed line represents rigidity of the overtube 40. At the same x coordinate, a value obtained by integrating rigidity of the insertion portion 2 and rigidity of the overtube 40 represents rigidity of the insertion portion 2 of the endoscope system 50 at the x coordinate.

FIG. 6 illustrates a state in which the distal end portion of the rigidity changing region 2a is exposed from the overtube 40 in the distal end direction and the rigidity changing mechanism 20 has not performed an operation of increasing rigidity of the flexible tube 10 yet. In other words, in the state shown in FIG. 6, the value of an x coordinate xC at the distal end of the overtube 40 is larger than L0−L1.

As shown in FIG. 6, in a region where the overtube 40 is externally fitted (x≥xC), since rigidity St of the cylindrical portion 41 of the overtube 40 is combined with rigidity of the insertion portion 2, rigidity becomes higher.

Note that although rigidity of the insertion portion 2 of the endoscope is assumed to have a constant value I1 regardless of the x coordinate in FIG. 6 for the sake of description, the rigidity of the insertion portion 2 may change in accordance with the x coordinate. The same applies to FIG. 7, FIG. 8 and FIG. 9.

FIG. 7 illustrates a state in which the distal end portion of the rigidity changing region 2a is exposed from the overtube 40 in the distal end direction and an operation to increase the rigidity of the flexible tube 10 by the rigidity changing mechanism 20 is in progress.

In the state shown in FIG. 7, the rigidity of the rigidity changing region 2a of the insertion portion 2 is increasing. The rigidity changing region 2a is a region where the x coordinate is larger than L0−L1. In the state shown in FIG. 7, the distal end of the overtube 40 is located within the rigidity changing region 2a.

In other words, the region where the overtube 40 is externally fitted and rigidity thereby increases is located closer to the proximal end side than the distal end of the rigidity changing region 2a. For this reason, in the state shown in FIG. 7, rigidity is lowest in the first region (x<(L0−L1)) on the distal end side compared to the rigidity in the rigidity changing region 2a, rigidity is intermediate in a second region ((L0−L1)≤x<xC) exposed from the overtube 40 in the distal end direction of the rigidity changing region 2a and rigidity is highest in a third region (x≥xC) covered with the overtube 40 of the rigidity changing region 2a.

The first region, the second region and the third region in which rigidity increases in this order, are disposed in order from the distal end of the insertion portion 2 toward the proximal end. Therefore, rigidity of the insertion portion 2 increases gently from the distal end toward the proximal end in the state shown in FIG. 7. Since the slope of a change in rigidity from the distal end of the insertion portion 2 toward the proximal end becomes gentle, insertability at the time of insertion into the subject of the insertion portion 2 can be improved.

FIG. 8 illustrates a state in which the distal end portion of the rigidity changing region 2a is covered with the overtube 40 and the rigidity changing mechanism 20 has not performed an operation of increasing rigidity of the flexible tube 10 yet. In other words, in the state shown in FIG. 7, the value of the x coordinate xC at the distal end of the overtube 40 is smaller than L0−L1.

FIG. 9 illustrates a state in which the distal end portion of the rigidity changing region 2a is covered with the overtube 40 and the operation of increasing rigidity of the flexible tube 10 by the rigidity changing mechanism 20 is in progress.

In the present embodiment, an increase St in rigidity by externally fitting the overtube 40 is set to be equal to an increase in rigidity of the flexible tube 10 by the rigidity changing mechanism 20. Therefore, when the distal end of the overtube 40 is located closer to the distal end side than the rigidity changing region 2a in the present embodiment, the rigidity of the flexible tube 10 can be increased without the rigidity changing mechanism 20 performing operation of increasing rigidity as shown in FIG. 8.

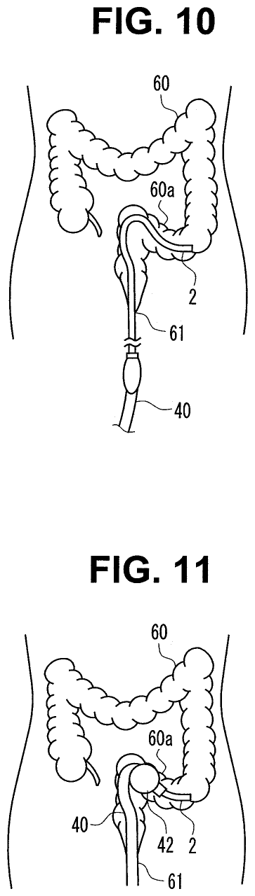

Next, a method of inserting the insertion portion 2 of the endoscope 1 into the large intestine 60 of a human body, which is the subject, via the anus 61 using the endoscope system 50 of the present embodiment will be described using FIG. 10 to FIG. 17.

In a first step, as shown in FIG. 10, only the insertion portion 2 of the endoscope 1 is inserted from the anus 61 without the rigidity changing mechanism 20 performing an operation of increasing rigidity of the flexible tube 10 until the distal end arrives at the sigmoid colon 60a of the large intestine 60 first. At this time, the overtube 40 is externally fitted onto the insertion portion 2 and the overtube 40 is kept pulled to the proximal end 10b side (operation portion 3 side) of the flexible tube 10. In other words, the overtube 40 is located outside the anus 61.

In the first step, within the range in which the insertion portion 2 is inserted into the large intestine 60, the operation of increasing rigidity of the flexible tube 10 by the rigidity changing mechanism 20 is not in progress and the insertion portion 2 is not covered with the overtube 40, and so the rigidity of the flexible tube 10 is lowest. Therefore, it is possible to easily advance the insertion portion 2 inside the sigmoid colon 60a with many bends.

Next, in a second step, as shown in FIG. 11, the overtube 40 is moved along the insertion portion 2 in the distal end direction, and the distal end of the overtube 40 is caused to arrive at the sigmoid colon 60a. Here, the overtube 40 is located closer to the distal end side than the distal end of the rigidity changing mechanism 20. As shown in FIG. 8, this operation causes the rigidity of the insertion portion 2 covered with the overtube 40 to increase.

A gas is sent from the balloon vent 43 into the balloon 42 to inflate the balloon 42 and fix the position of the overtube 40.

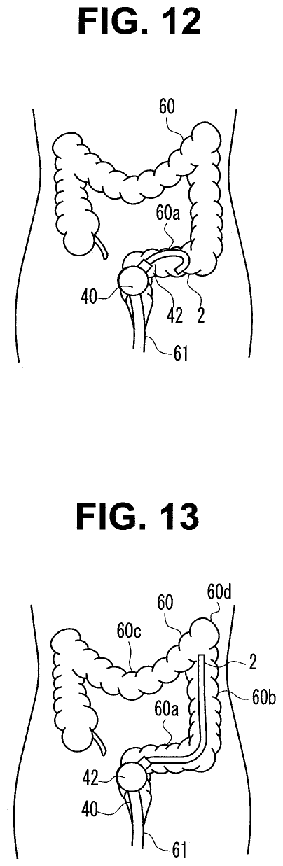

Next, in a third step, as shown in FIG. 12, by pulling the overtube 40, the position of which is fixed and the insertion portion 2, rigidity of which has increased, the sigmoid colon 60a is straightened. Furthermore, the rigidity changing mechanism 20 performs an operation of increasing rigidity of the flexible tube 10.

Next, in a fourth step, as shown in FIG. 13, with the position of the overtube 40 fixed, the insertion portion 2, rigidity of which has been increased is pushed in and the distal end of the insertion portion 2 is caused to advance to the splenic flexure 60d between the descending colon 60b and the transverse colon 60c. At this time, as shown in FIG. 7, the rigidity of the insertion portion 2 increases gently from the distal end toward the proximal end. In other words, it is possible to easily advance the distal end side of the insertion portion 2, rigidity of which is low, while keeping the rigidity of the portion of the insertion portion 2 on the proximal end side inserted into the straightened sigmoid colon 60a.

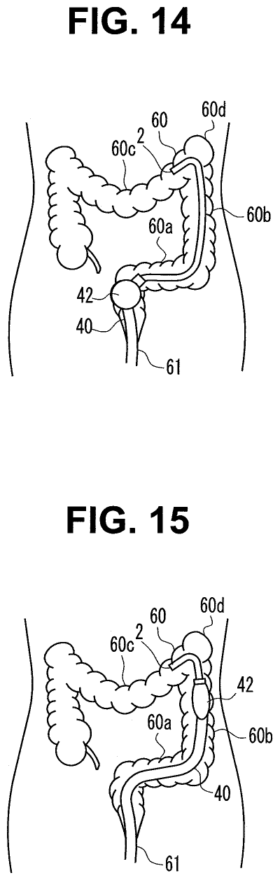

In a fifth step, as shown in FIG. 14, the distal end of the insertion portion 2 is caused to advance into the transverse colon 60c.

Next, in a sixth step, as shown in FIG. 15, after deflating the balloon 42, the overtube 40 is caused to move along the insertion portion 2 in the distal end direction, and the distal end of the overtube 40 is caused to advance to the splenic flexure 60d. The operation of increasing rigidity of the flexible tube 10 by the rigidity changing mechanism 20 is not performed.

At this time, as shown in FIG. 8, without the rigidity changing mechanism 20 performing an operation of increasing rigidity, the rigidity of the flexible tube 10 remains increased with the presence of the overtube 40. Therefore, the shape of the straightened sigmoid colon 60a is kept.

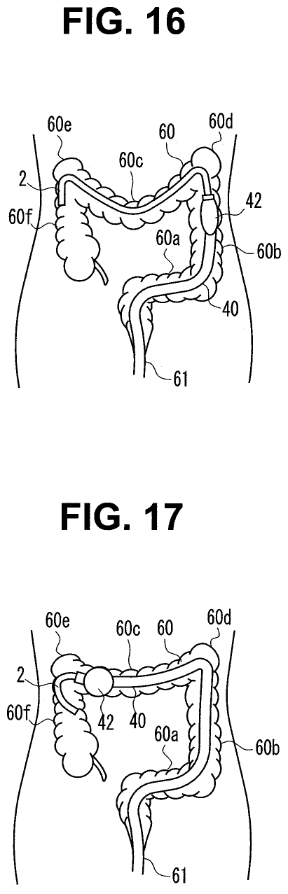

Next, in a seventh step, as shown in FIG. 16 without the rigidity changing mechanism 20 performing an operation of increasing rigidity, with the position of the overtube 40 fixed, only the insertion portion 2 is caused to advance until the distal end arrives at the hepatic flexure 60e. At this time, the shape of the sigmoid colon 60a straightened due to the rigidity of the overtube 40 is kept and the range in which the insertion portion 2 is inserted in the large intestine 60 is a range in which the rigidity changing mechanism 20 does not perform any operation of increasing rigidity of the flexible tube 10 and the flexible tube 10 is not covered with the overtube 40, is least rigid and flexible, and so it is possible to easily advance the insertion portion 2 even inside the transverse colon 60c which has many bends and which is not fixed (if the insertion portion 2 remains rigid, the bent portions of the transverse colon are considerably drooped toward the anus side, and it is difficult to insert the insertion portion 2).

Next, in an eighth step, as shown in 17, the rigidity changing mechanism 20 performs an operation of increasing rigidity of the flexible tube 10 and lifts the transverse colon 60c. The overtube 40 is caused to move along the insertion portion 2 in the distal end direction, the distal end of the overtube 40 is caused to advance to the hepatic flexure 60e, and then the balloon 42 is inflated to fix the position of the overtube 40.

When the distal end of the overtube 40 is fixed to the vicinity of the hepatic flexure 60e, the shapes of the straightened sigmoid colon 60a and the lifted transverse colon 60c are kept, and so the operation of advancing the insertion portion 2 to the ascending colon 60f, which is located deeper in the large intestine, becomes easy.

As described so far, when the endoscope system 50 of the present embodiment makes various rigidity settings by combining switching whether or not to perform an operation of increasing rigidity of the flexible tube 10 by the rigidity changing mechanism 20 and moving the overtube 40 forward or backward in the longitudinal direction relative to the insertion portion 2, it is possible to easily confirm a positional relationship between the distal end of the rigidity changing region and the overtube 40.

In the aforementioned embodiments, the large diameter portion 10c having an outer diameter larger than the outer diameter of the flexible tube 10 is provided at a position corresponding to the distal end portion of the rigidity changing region of the flexible tube 10 as the notification unit. As other means of the notification unit, for example, an index may be provided at a portion of the overtube 40 where the proximal end portion 41b of the overtube 40 is located when the distal end portion 41a of the overtube 40 is located at the distal end of the rigidity changing region on the proximal end side of the flexible tube 10. Since this index is always located outside a patient's body, the operator can easily visually recognize the index.

The present invention is not limited to the aforementioned embodiments, but can be changed as appropriate without departing from the scope of the gist or thought of the present invention that can be read from the scope of claims and the entire specification, and such a changed endoscope system is also included in the technical scope of the present invention.

What is claimed is:

1. endoscope comprising:
an elongated insertion portion;
a flexible tube included in a proximal end side of the insertion portion;
a rigidity changing mechanism provided inside the flexible tube and configured to change rigidity of the flexible tube by an operation on a hand side; and
an index provided on an outer circumference of the flexible tube, configured to indicate a distal end of a rigidity changing region of the flexible tube by the rigidity changing mechanism and made up of a large diameter portion having an outer diameter larger than an outer diameter of the flexible tube.

2. The endoscope according to claim 1, wherein a rigidity changing region of the flexible tube by the rigidity changing mechanism is set to extend from a middle portion of the flexible tube to a proximal end portion.

3. The endoscope according to claim 1, wherein a flexible, axially extending and cylindrical overtube is slidably attached to an outside of the flexible tube, an overall length in an axial direction of the overtube being set to be shorter than an overall length in an axial direction of the rigidity changing region.

4. The endoscope according to claim 3, wherein the outer diameter of the large diameter portion is set to be larger than an inner diameter of the overtube at the distal end portion and smaller than an outer diameter of the overtube.

5. The endoscope according to claim 3, wherein a locking portion configured to contact a proximal end portion of the overtube and restrict movement of the overtube toward a hand side is provided at a proximal end portion of the flexible tube.

6. An endoscope system comprising:
an endoscope including an elongated insertion portion, a flexible tube included in a proximal end side of the insertion portion, and a rigidity changing mechanism provided inside the flexible tube and configured to change rigidity of the flexible tube, a rigidity changing region of the flexible tube being set by the rigidity changing mechanism so as to extend from a middle portion to a proximal end portion of the flexible tube;
a flexible, axially extending and cylindrical overtube, into which the insertion portion is slidably inserted, an overall length in an axial direction of the overtube being formed to be shorter than an overall length of the rigidity changing region, the overtube being configured to expose a distal end portion of the rigidity changing region when a proximal end portion of the overtube in the axial direction is positioned on a most proximal end side of the insertion portion of the endoscope; and
a notification unit provided at a position corresponding to the distal end portion of the rigidity changing region in the flexible tube, made up of a large diameter portion having an outer diameter larger than an outer diameter of the flexible tube and configured to notify that the distal end of the overtube passes through the distal end of the rigidity changing region.

7. The endoscope system according to claim 6, wherein the outer diameter of the large diameter portion is set to be larger than an inner diameter of the overtube at the distal end portion and smaller than an outer diameter of the overtube.

8. The endoscope system according to claim 6, wherein a locking portion configured to contact the proximal end portion of the overtube and restrict movement of the overtube toward a hand side is provided at the proximal end portion of the flexible tube.

9. The endoscope system according to claim 6, wherein the notification unit is an index provided at a portion through which the proximal end portion of the overtube passes when the distal end of the overtube of the flexible tube passes through the distal end of the rigidity changing region.
